(19) United States
(12) Patent Application Publication
(10) Pub. No.: US 2026/0265175 A1
(43) Pub. Date: Sep. 10, 2026

(54) METHOD FOR OBTAINING ORGANIC ACIDS FROM AN AQUEOUS MOTHER LIQUOR

(71) Applicant: Covestro Deutschland AG, Leverkusen (DE)

(72) Inventors: Eric Ritter, Köln (DE); Wolf Kloeckner, Köln (DE); Dirk Kreyenschulte, Langenfeld (DE); Martin Machat, Köln (DE); Melina Sandfort, Dormagen (DE)

(21) Appl. No.: 18/871,845

(22) PCT Filed: Jun. 27, 2023
(86) PCT No.: PCT/EP2023/067436

§ 371 Date: Dec. 5, 2024

(30) Foreign Application Priority Data
Jun. 30, 2022 (EP) 22182138.2
Jun. 13, 2023 (EP) 23179004.9

(51) Int. Cl. C07C 227/42; C07C 51/43; C07C 51/47

(52) U.S. Cl. CPC C07C 227/42; C07C 51/43; C07C 51/47

(57) ABSTRACT

The invention relates to a method for obtaining an aminobenzoic acid or an aliphatic saturated C₄₋₆-dicarboxylic acid from an aqueous solution, comprising: A) producing the organic acid, wherein a) the organic acid is crytallised from an aqueous medium, followed by a separation of the precipitated portions of the organic acid via a solid-liquid phase separation, leaving a first aqueous solution of the organic acid; B) treating the aqueous solution of the organic acid with an Mn²⁺, Fe²⁺ and/or Cu²⁺ metal salt, precipitating a metal compound of the organic acid, followed by a separation of the precipitated metal compound via a solid-liquid phase separation; C) carrying out a base treatment of the separated metal compound, precipitating metal hydroxide and separating precipitated metal hydroxide via a solid-liquid phase separation, leaving a basic aqueous solution containing anions of the organic acid; and D) crystallising organic acid from the basic aqueous solution obtained in C) by adding an inorganic acid.

The invention on which this application is based was financially supported by the German Federal Ministry of Food and Agriculture as part of the project “Biobasierte Herstellung von Intermediaten für Polyurethane—Phase II (Bio4PURPro)” [“Biobased Preparation of Intermediates for Polyurethanes—Phase II (Bio4PURPro)”] (funding code 22019918).

The present invention relates to a process for obtaining an organic acid from an aqueous solution of the organic acid, wherein the organic acid is selected from an optionally methyl-substituted aminobenzoic acid or an aliphatic saturated C₄ to C₆ dicarboxylic acid, said process comprising the following: A) preparation of the organic acid by a biological process or a chemical reaction, wherein a) the organic acid is crystallized from an aqueous medium during or after the preparation, followed by 1) removing the fractions of the organic acid that have precipitated in the crystallization by a solid-liquid phase separation to leave a first aqueous solution of the organic acid, and optionally 2) depleting the fraction of the organic acid dissolved in the first aqueous solution of the organic acid by extracting organic acid with an organic extractant or by adsorbing organic acid on an adsorbent, followed by removing of the organic extractant or adsorbent to obtain a second aqueous solution of the organic acid; B) treating the first aqueous solution of the inorganic acid or the second aqueous solution of the organic acid with a metal salt, the metal ions of which include Mn²⁺, Fe²⁺ and/or Cu²⁺, to precipitate a metal compound of the organic acid, followed by removing of the precipitated metal compound of the organic acid by a solid-liquid phase separation; C) base treatment of the metal compound of the organic acid that has been removed with an aqueous base solution with precipitation of metal hydroxide and removal of precipitated metal hydroxide by a solid-liquid phase separation to leave a basic aqueous solution containing anions of the organic acid; and D) crystallizing organic acid out of the basic aqueous solution containing anions of the organic acid that has been obtained in C) by addition of an inorganic acid.

A recurring feature of the preparation of organic acids is also crystallization steps from an aqueous medium for isolation and/or purification of the inorganic acid, in some cases from solutions containing the anion of the organic acid by lowering the pH (called reactive crystallization). Removal of the crystallized organic acid leaves an aqueous solution of the organic acid (=the mother liquor from the crystallization) which still includes a residual content of organic acid corresponding to the solubility equilibrium, which may quite possibly be in the region of a few grams per liter.

One example of this is the preparation of anthranilic acid. Anthranilic acid finds use, for example, in the production of dyes, odorants or pharmaceuticals. The preparation of heterocycles from anthranilic acid and its derivatives is the subject of the thesis Synthesis of Heterocycles from Anthranilic acid and its Derivatives by Per Wiklund, Karolinska University Press 2004, ISBN 91-7349-913-7. In Separation and Determination of Iron(II) and Iron(III) with Anthranilic Acid Using Solvent Extraction and Spectrophotometry, published in Anal. Chem. 1963, 35, 2077-2081, Donald L. Dinsel and Thomas R. Sweet describe the use of anthranilic acid as extractant for iron salts. The reduction of iron(III) by anthranilic acid is addressed by Alexander A. Kamnev and Erno Kuzmann in Mössbauer spectroscopic evidence for the reduction of iron(III) by anthranilic acid in aqueous solution, published in Polyhedron 1997, 16, 3353-3356. The redox chemistry and iron complexation of anthranilic acid and of 3-hydroxyanthranilic acid is the subject of the paper Iron chelation and redox chemistry of anthranilic acid and 3-hydroxyanthranilic acid: A comparison of two structurally related kynurenine pathway metabolites to obtain improved insights into their potential role in neurological disease development by V. Chobot, F. Hadacek, W. Weckwerth and L. Kubicova, published in J. Organomet. Chem. 2015, 782, 103-110.

A further example of a use of anthranilic acid is the use thereof in the production of aniline by decarboxylation (as described, for example, in WO 2015/124687 A1). Aniline in turn is of particular significance as an intermediate in the production of isocyanates. Anthranilic acid can be obtained, for example, by fermentation (see, for instance, the already mentioned publication WO 2015/124687 A1) or chemically (for example by Hoffman rearrangement of phthalimide; see, for instance, WO 2007/088346 A1). Irrespective of the exact manner of production, crystallization steps of the abovementioned type are regularly conducted. Mention is made by way of example of the isolation of anthranilic acid from fermentation broths containing the anion of anthranilic acid by reactive crystallization (again see WO 2015/124687 A1). Anthranilic acid is an example of an aminobenzoic acid. Further important representatives of this compound class are anthranilic acid derivatives in which a hydrogen atom in the benzene ring has been replaced by a methyl group. The complex formation properties of aminobenzoic acids having substitution on the benzene ring is addressed by J. S. Decker and H. Frye in the article Metal Ion Complexes of Aromatic Amino Acids—I. Ring Substituted Amino Benzoic Acids, in Z. Naturforschg. 1966, 21 b, 522-526.

A further example is adipic acid. Adipic acid is an intermediate in the production of nylon. Adipic acid is also used as a precursor for the synthesis of polyester polyols for polyurethane products. Adipic acid can also be obtained by fermentation (see, for instance, Peter C. K. Lau (Editor), Quality Living Through Chemurgy and Green Chemistry, Springer-Verlag GmbH Germany 2016, chapter 3.3, pages 47 to 54, and K. Raj, S. Partow et al., Biocatalytic production of adipic acid from glucose using engineered Saccharomyces cerevisiae in Metabolic Engineering Communications 2018, 6, 28-32), or chemically (for example by hydrogenation of muconic acid; see, for instance, WO 2015/086827 A1, or by oxidation of cyclohexane; see, for instance, A. Castellan et al., Industrial production and use of adipic acid in Catal. Today 1991, 9, 237-254). Here too, crystallization steps of the abovementioned type regularly occur. Reference is made by way of example to the purification of the reaction product from a muconic acid hydrogenation by crystallization (see WO 2015/086827 A1). Adipic acid is an example of an aliphatic saturated dicarboxylic acid. A further important representative of this compound class is succinic acid.

It is therefore important to reduce the residual content of organic acid in the mother liquor by suitable methods in order to avoid yield losses and to minimize wastewater pollution.

EP 0 502 384 A2 describes a process for recovering adipic acid from aqueous nitric acid-containing mother liquors obtained in industrial adipic acid production by selective crystallization of the dissolved adipic acid, in which the mother liquor is admixed within the temperature range from 30 to 60° C. with such an amount of an aqueous adipic acid solution having an adipic acid content of 0.5% to 6% by weight that the nitric acid content of the mixture is lowered to 35% to 50% by weight, then the mixture is cooled by at least 5° C. within a period of 0.5 to 5 hours, the adipic acid that crystallizes out here is isolated by filtration and the resultant filtrate is sent to distillative glutaric acid workup.

WO 2015/085198 A1 describes a process for producing succinic acid and succinic esters from a succinic salt in a fermentation broth. In a first stage, renewable carbon resources are used to produce succinic acid by biological fermentation. The succinic salt is subjected to a double displacement reaction with a strong acid in the fermentation process, which leads to release of succinic acid. Succinic acid is obtained by fractional crystallization, integrated with SMB (simulated moving bed) chromatography, in order to produce succinic acid and succinic esters.

International application WO 2015/124687 A1, which has already been mentioned several times, discloses a preferred embodiment in this regard, in which the mother liquor obtained in the crystallization is worked up in order to obtain further aminobenzoic acid. This is accomplished by a sequence composed of an adsorption step and a desorption step. The aminobenzoic acid-enriched desorbate obtained is recycled into the crystallization procedure. In this way, yield losses are reduced. The adsorption is effected onto zeolites or activated carbon, and the desorption with water of a pH in the range from 5 to 10 or, alternatively, with organic solvents, especially 1-dodecanol. A variant of such a workup by a sequence of an adsorption step and desorption step is described in international application WO 2018/114841 A1. The process disclosed therein is more particularly characterized in that the desorption is conducted in an acidic environment (pH −0.8 to 3.0).

International patent application WO 2007/088346 A1, which has likewise already been mentioned, describes a process for recovering anthranilic acid from the mother liquor from a crystallization for isolation of anthranilic acid that has been obtained by a Hofmann rearrangement. The sodium carbonate-containing product of the Hofmann rearrangement is adjusted to a pH of 4.2 with sulfuric acid (a), and precipitated anthranilic acid is filtered off (b). The remaining mother liquor is extracted with an organic solvent at a pH of 4.2; suitable organic solvents disclosed are acetic esters (especially ethyl or butyl acetate), ketones (especially 2-butanone) and aromatic hydrocarbons (especially toluene). The organic extract obtained after phase separation (c) is subsequently re-extracted with sodium hydroxide solution (d), with transfer of anthranilic acid as anthranilate anion into the aqueous phase obtained after phase separation (e). This aqueous, anthranilic acid-enriched phase from the re-extraction is combined with the product of the Hofmann rearrangement and sent to the crystallization together therewith. The aqueous, anthranilic acid-depleted phase obtained in the extraction is adjusted to pH 1.5 with sulfuric acid, an organic solvent is added (f), and the mixture is separated into an aqueous phase and an organic phase (g). In this way, organic impurities are transferred to the organic phase. This organic phase is combined with the organic phase from the re-extraction and distilled. The distillate can be recycled into the process as solvent. The distillation residue is incinerated. The aqueous phase from (g) is sent to the wastewater after purification by a Fenton reaction. A particular disadvantage of this process is the relatively high water solubility of the acetic esters and ketones. Acetic esters and ketones do dissolve anthranilic acid efficiently, but get into the aqueous phase in non-negligible proportions because of their comparatively high water solubility. This firstly results in yield losses and secondly increases complexity in the cleaning of wastewater. Toluene, which is likewise disclosed as a suitable solvent in WO 2007/088346 A1, does not have the disadvantages described, but dissolves anthranilic acid considerably less efficiently, which makes it difficult to develop a practicable process on an industrial scale with toluene as extractant.

CN 102 190 590 A describes a process for removing anthranilic acid from the wastewaters from methyl anthranilate production. In the process described, the wastewater from methyl anthranilate production is admixed with acidic wastewater (in particular copper chloride-containing sulfuric acid) until a pH of 3.5 to 4.5 is established, and copper anthranilate is precipitated. The precipitated copper anthranilate is added to a stirred tank together with calcium oxide and admixed with alkaline solution (pH 11 to 13). The mixture is heated to 90 to 110° C. and kept at boiling for 30 to 150 minutes. After filtration, the sodium anthranilate-containing filtrate is decolorized and adjusted to a pH of 3.5 to 4.0 with hydrochloric acid. Precipitated anthranilic acid is washed and dried.

None of the prior art processes described is free of disadvantages, and so there was a need for further improvements in the field of recovery of organic acids from aqueous solutions thereof (mother liquors). In particular, a desirable process would be one in which, firstly, the organic acid can be removed selectively and virtually completely from the aqueous mother liquor without transfer of other components, for example an organic solvent, into the aqueous phase. Secondly, a desirable process would be one in which the organic acid removed can be transferred back to an aqueous phase with high concentration and yield.

Taking account of this requirement, the present invention provides a process for obtaining an organic acid from an aqueous solution of the organic acid, where the organic acid is selected from
(i) an aminobenzoic acid of the formula

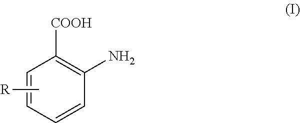

in which R is CH₃ or H, or
(ii) an aliphatic saturated dicarboxylic acid of the formula

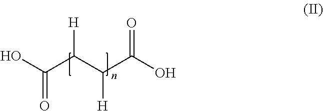

in which n=1 or—preferably—2,
said process comprising the steps of:
A) preparation of the organic acid by a biological process (preferably a fermentation) or a chemical reaction (i.e. in a nonbiological process), wherein
   α) the organic acid is crystallized from an aqueous medium during or after the preparation (in order to isolate it, optionally with protonation of the anion of the organic acid, or in order to purify already isolated organic acid by recrystallization),
   followed by
   1) removing the fractions of the organic acid that have precipitated in the crystallization (i.e. in the crystallization of step a)) by a solid-liquid phase separation to leave a first aqueous solution of the organic acid and
   2) optionally depleting the fraction of the organic acid dissolved in the first aqueous solution of the organic acid
      by extracting organic acid with an organic extractant or
      by adsorbing organic acid on an adsorbent,
      followed by removing of the (organic acid-laden) organic extractant or adsorbent to obtain an (organic acid-depleted) second aqueous solution of the organic acid;
B) treating the first aqueous solution of the organic acid or the second aqueous solution of the organic acid with a metal salt, the metal ions of which include Mn²⁺ Fe²⁺ and/or Cu²⁺ (and are especially selected from the group consisting of Mn²⁺, Fe²⁺ and Cu²⁺), to precipitate a metal compound of the organic acid (sparingly soluble complex compound or low-solubility salt),
   followed by removing of the precipitated metal compound of the organic acid by a solid-liquid phase separation;
C) base treatment of the metal compound of the organic acid that has been removed with an aqueous base solution with precipitation of metal hydroxide and removal of precipitated metal hydroxide by a solid-liquid phase separation to leave a basic aqueous solution containing anions of the organic acid, wherein the base treatment is especially conducted without use of calcium oxide and especially at a temperature of 20° C. to 85° C., preferably 20° C. to 70° C., more preferably 20° C. to 50° C., especially also at ambient temperature;
and
D) crystallizing organic acid out of the basic aqueous solution containing anions of the organic acid that has been obtained in C) by addition of an inorganic acid, especially hydrochloric acid, sulfuric acid, nitric acid or phosphoric acid.

Completely surprisingly, it has been found that it is possible by the combination of treatment with a metal salt of the type specified (step B)) and base treatment (step C)) to provide an aqueous solution of the organic acid (in anionic form) which is of much higher concentration than the aqueous solution of the organic acid that originates from A)α) and which, as a result, can be sent to a reactive crystallization (step D)) in order to isolate further organic acid.

All pH values in the context of the present invention relate to the temperature at which the corresponding step (e.g. step (D)) is conducted and can simply be measured with a glass electrode.

Biological methods in the context of the present invention are fermentative or enzymatic conversions of organic substances.

Accordingly, in the context of the present invention, the conversion of an organic substance is referred to as chemical (chemical conversion or chemical method) if it proceeds without use of fermentative or enzymatic processes (i.e. purely thermally or using non-biological catalysts).

The appended figures show:

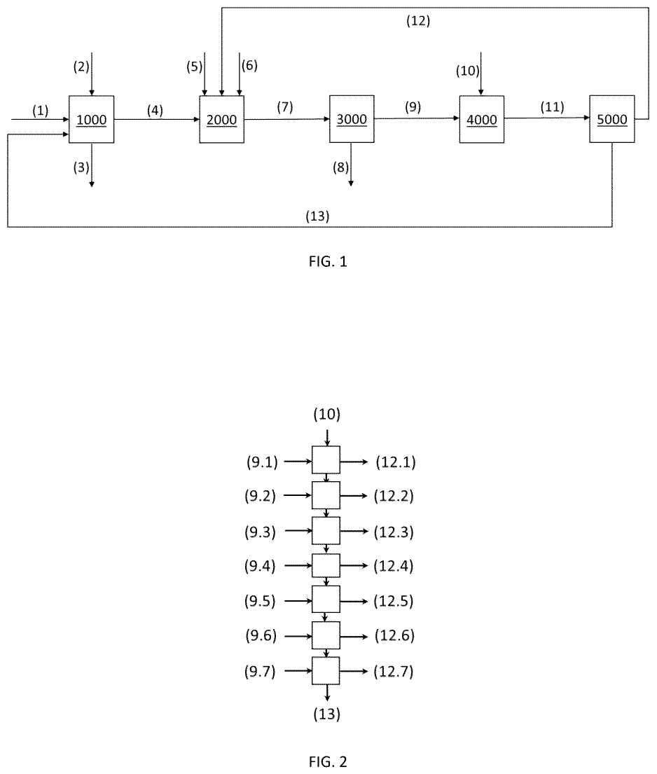

FIG. 1 a schematic diagram of one embodiment of the process of the invention; and

FIG. 2 a schematic diagram of the multistage performance of step C).

There will firstly follow a summary of various possible embodiments of the invention:

In a first embodiment of the invention, which can be combined with all other embodiments, the base treatment in C) comprises n steps conducted in succession, where n is a natural number in the range from 2 to 10, preferably 3 to 9, more preferably 4 to 8, wherein
in each of the n steps, a portion of the metal compound of the organic acid that has been removed is treated with an aqueous base solution with precipitation of metal hydroxide, wherein each step is followed by removal of precipitated metal hydroxide by solid-liquid phase separation to leave a basic aqueous solution containing anions of the organic acid, wherein the aqueous base solution in the second to nth step comprises the basic aqueous solution containing anions of the organic acid that has been obtained in the respective preceding step (and optionally consists thereof, i.e. does not comprise any further constituents);
wherein the crystallizing of the organic acid in D) is conducted with the basic aqueous solution containing anions of the organic acid that has been obtained in the nth step of C).

In a second embodiment of the invention, which can be combined with all other embodiments, provided that these do not envisage separate performance of the crystallization of the organic acid in D) and in A)α), the crystallizing of organic acid in D) comprises the recycling of the basic aqueous solution containing anions of the organic acid which is obtained in C) (i.e. in the nth step of C) in the case of n-stage base treatment thereof) to A)α).

In a third embodiment of the invention, which can be combined with all other embodiments, provided that these do not envisage separate performance of the crystallization of the organic acid in D) and in A)α), the crystallizing of organic acid in D) is conducted separately from A)α).

In a fourth embodiment of the invention, which can be combined with all other embodiments, the metal hydroxide precipitated in C) is recycled into step B), where it is used as a constituent of the metal salt.

In a fifth embodiment of the invention, which can be combined with all other embodiments, provided that these do not relate exclusively to aliphatic saturated dicarboxylic acid of the formula (II), the organic acid is an aminobenzoic acid of the formula (I), especially 2-aminobenzoic acid (anthranilic acid) or 2-amino-5-methylbenzoic acid, preferably 2-aminobenzoic acid.

In a sixth embodiment of the invention, which is a particular configuration of the fifth embodiment, a pH in the range from 3.0 to 4.7, preferably 3.2 to 3.7, more preferably 3.4 to 3.6, is established in A)α) by addition of an inorganic acid, especially hydrochloric acid, sulfuric acid, nitric acid or phosphoric acid, and a pH within the same range is maintained in B) (optionally by adding an acid or base if the pH should rise or fall too much as a result of the addition of the metal salt).

In a seventh embodiment of the invention, which is a particular configuration of the fifth and sixth embodiments, step A) comprises a biological method, namely the fermentation of a raw material containing a fermentable carbon-containing compound and a nitrogen-containing compound in the presence of microorganisms.

In an eighth embodiment of the invention, which is a particular configuration of the seventh embodiment,
(I) A)α) is performed by conducting the fermentation at a pH in the range from 3.0 to 4.7, preferably 3.2 to 3.7, more preferably 3.4 to 3.6, such that the aminobenzoic acid of the formula (I) already crystallizes in the fermentation;
or
(II) the fermentation is conducted at a pH in the range from >4.7 to 11 (especially 6.0 to 11), preferably 4.8 to 9.0 (especially 6.0 to 9.0), more preferably 5.0 to 8.0 (especially 6.0 to 8.0), where A)α) is performed by establishing, after the fermentation, a pH in the range from 3.0 to 4.7, preferably 3.2 to 3.7, more preferably 3.4 to 3.6.

In a ninth embodiment of the invention, which is a particular configuration of the seventh and eighth embodiments, the microorganisms are selected from Escherichia coli, Pseudomonas putida, Corynebacterium glutamicum, Ashbya gossypii, Pichia pastoris, Hansenula polymorpha, Kluyveromyces marxianus, Yarrowia lipolytica, Zygosaccharomyces bailii or Saccharomyces cerevisiae.

In a tenth embodiment of the invention, which is a particular configuration of the fifth and sixth embodiments, A) comprises a chemical conversion (i.e. a non-biological method).

In an eleventh embodiment of the invention, which is a particular configuration of the tenth embodiment, A) comprises the conversion of optionally methyl-substituted phthalimide or phthalamide with an alkali metal hypohalite (especially sodium hypochlorite) in a basic medium, followed by the addition of an inorganic acid, especially hydrochloric acid, sulfuric acid, nitric acid or phosphoric acid, for performance of A)α).

In a twelfth embodiment of the invention, which can be combined with all other embodiments, provided that these do not relate exclusively to aminobenzoic acids of the formula (I), the organic acid is an aliphatic saturated dicarboxylic acid of the formula (II), especially hexane-1,6-dicarboxylic acid (adipic acid).

In a thirteenth embodiment of the invention, which is a particular configuration of the twelfth embodiment, a pH in the range from 0 to 4.0, preferably 1.8 to 3.0, more preferably 2.0 to 2.5, is established in A)α) by addition of an inorganic acid, especially hydrochloric acid, sulfuric acid, nitric acid or phosphoric acid, and a pH in the range from >4.0 to 7.0, preferably 4.7 to 5.8, more preferably 5.3 to 5.7, is established in B) by addition of a base.

In a fourteenth embodiment of the invention, which is a particular configuration of the twelfth and thirteenth embodiments, step A) comprises a biological method.

In a fifteenth embodiment of the invention, which is a particular configuration of the fourteenth embodiment, the biological method comprises the fermentation of a raw material containing a fermentable carbon-containing compound in the presence of microorganisms.

In a sixteenth embodiment of the invention, which is a particular configuration of the fifteenth embodiment,
(I) A)α) is performed by conducting the fermentation at a pH in the range from 0 to 4.0, preferably 1.8 to 3.0, more preferably 2.0 to 2.5, such that the aliphatic saturated dicarboxylic acid of the formula (I) already crystallizes in the fermentation;
or
(II) the fermentation is conducted at a pH in the range from >4.0 to 11 (especially 6.0 to 11), preferably 5.0 to 9.0 (especially 6.0 to 9.0), more preferably 5.0 to 8.0 (especially 6.0 to 8.0), where A)α) is performed by establishing, after the fermentation, a pH in the range from 0 to 4.0, preferably 1.8 to 3.0, more preferably 2.0 to 2.5.

In a seventeenth embodiment of the invention, which is a particular configuration of the fifteenth and sixteenth embodiments, the microorganisms are selected from Escherichia coli, Pseudomonas putida, Corynebacterium glutamicum, Bacillus coagulans, Ashbya gossypii, Pichia pastoris, Hansenula polymorpha, Kluyveromyces marxianus, Yarrowia lipolytica, Zygosaccharomyces bailii or Saccharomyces cerevisiae.

In an eighteenth embodiment of the invention, which is a particular configuration of the twelfth and thirteenth embodiments, A) comprises a chemical conversion (i.e. a non-biological method).

In a nineteenth embodiment of the invention, which is a particular configuration of the eighteenth embodiment, it is especially the case that n=2 (i.e. the organic acid is thus in particular adipic acid), wherein A) comprises the hydrogenation of an aliphatic unsaturated dicarboxylic acid corresponding to the aliphatic saturated dicarboxylic acid of the formula (II) (such as in particular muconic acid) (and A)α) is conducted especially for purification of the product obtained in the hydrogenation).

In a twentieth embodiment of the invention, which is a further particular configuration of the eighteenth embodiment, the aliphatic saturated dicarboxylic acid of the formula (II) is hexane-1,6-dicarboxylic acid (adipic acid), where A) comprises the oxidation of cyclohexane with oxygen or oxygen-containing gas mixtures (especially air) to give cyclohexanol and cyclohexanone and subsequent oxidation thereof with nitric acid.

In a twenty-first embodiment of the invention, which is a particular configuration of the fourteenth and eighteenth embodiments, A) comprises an (enzymatic or chemical) cleavage of a polyester.

In a twenty-second embodiment of the invention, which is combinable with all other embodiments, especially to the extent that these relate to the obtaining of an aliphatic saturated dicarboxylic acid of the formula (II) (preferably hexane-1,6-dicarboxylic acid, adipic acid), the metal ions of the metal salt used in B) comprise Fe²⁺ and/or Cu²⁺ and are especially selected from the group consisting of Fe²⁺ and Cu²⁺ (where, especially in the case of the obtaining of an aliphatic dicarboxylic acid of the formula (II), Cu²⁺ is preferred).

In a twenty-third embodiment of the invention, which is combinable with all other embodiments, a pH in the range from 9.0 to 14, preferably 10 to 14, more preferably 11 to 13, is established in C) (in the case of n-stage base treatment in each of the n steps of the base treatment).

In a twenty-fourth embodiment of the invention, which is combinable with all other embodiments, the metal ions are added in B) in not more than a stoichiometric amount, based on the amount of the organic acid.

In a twenty-fifth embodiment of the invention, which is combinable with all other embodiments, the metal salt comprises a sulfate, chloride and/or hydroxide.

In a twenty-sixth embodiment of the invention, which is combinable with all other embodiments, the solid-liquid phase separations in A), B) and/or C) are conducted by filtration, centrifugation or sedimentation, optionally assisted by a preceding flocculation.

In a twenty-seventh embodiment of the invention, which is combinable with all other embodiments, step 2) is conducted in A), wherein
the organic extractant is selected from (i) an alcohol, especially an alkanol, having 4 to 12 carbon atoms (especially 1-decanol and/or 1-dodecanol), preferably 8 to 12 carbon atoms, more preferably 9 to 11 carbon atoms, (ii) a ketone (especially methyl iso-butyl ketone and/or cyclohexanone), (iii) an ether (especially diethyl ether), (iv) an ester (especially butyl acetate), (v) an aromatic, optionally halogen-substituted hydrocarbon (especially benzene, toluene, monochlorobenzene and/or dichlorobenzene) or (vi) a mixture of two or more of the aforementioned extractants,
and
the adsorbent is selected from (i), activated carbon, (ii), polymer-based adsorbents, (iii) graphite, (iv) a polar adsorbent such as, in particular, silica gel, limestone or an aluminum silicate, or (v) a mixture of two or more of the aforementioned adsorbents.

In a twenty-eighth embodiment of the invention, which is combinable with all other embodiments, the removal of the precipitated metal compound of the organic acid by solid-liquid phase separation in B) leaves an aqueous phase which is adjusted to a pH in the range from 8.0 to 14, preferably 10 to 13, by addition of a base, with precipitation of metal hydroxides that are removed by a further solid-liquid phase separation and then used as a constituent of the metal salt in B).

In a twenty-ninth embodiment of the invention, which is a particular configuration of the twenty-eighth embodiment, the base used to establish the pH comprises an alkali metal or alkaline earth metal hydroxide, an alkali metal or alkaline earth metal carbonate, an alkali metal or alkaline earth metal hydrogencarbonate, especially sodium hydroxide solution, potassium hydroxide solution or calcium hydroxide solution.

The embodiments briefly outlined above and further possible configurations of the invention are elucidated in detail hereinafter. All the above-described embodiments and the further configurations of the invention described below are mutually and collectively combinable as desired unless the opposite is clearly apparent from the context to a person skilled in the art or is expressly stated.

### Preparation of the Organic Acid

Step (A) of the process of the invention, the preparation of the organic acid, is of course dependent on the type of acid desired. During the preparation or thereafter, the crystallization step A)α) takes place.

### Aminobenzoic Acids of the Formula (I)

If the aim is to obtain aminobenzoic acid of the formula (I), of which 2-aminobenzoic acid (=anthranilic acid) is preferred, it has been found to be useful to establish a pH in the range from 3.0 to 4.7, preferably 3.2 to 3.7, more preferably 3.4 to 3.6, in step A)α) by addition of an inorganic acid, especially hydrochloric acid, sulfuric acid, nitric acid or phosphoric acid. The exact manner of performance of the crystallization in step A)α) is in turn dependent on the manner of performance of step A) overall.

### Biological Methods

In one embodiment, step A) comprises a fermentative step. This is conducted by converting a fermentable carbon-containing compound and a nitrogen-containing compound to the desired aminobenzoic acid or anion thereof in the presence of microorganisms. Such fermentations have already been described; see the literature cited hereinafter. Suitable microorganisms for a fermentative preparation of an aminobenzoic acid of the formula (I) include in particular Escherichia coli, Pseudomonas putida, Corynebacterium glutamicum, Ashbya gossypii, Pichia pastoris, Hansenula polymorpha, Kluyveromyces marxianus, Yarrowia lipolytica, Zygosaccharomyces bailii or Saccharomyces cerevisiae.

The fermentation in step A) is preferably conducted in such a way that the pH in the fermentation broth obtained is in the range from 3.0 to 11, preferably 4.7 to 11 (=case (II);

see below). If required, the pH can be controlled by addition of aqueous or gaseous ammonia, aqueous potassium hydroxide or aqueous sodium hydroxide (when pH values are too low), or by addition of a mineral acid, especially of hydrochloric acid, sulfuric acid, phosphoric acid or nitric acid (when pH values are too high). Different pH ranges within the pH range from 3.0 to 11 cited may be particularly optimal for different microorganisms.

The pH in the fermentation also has an influence on step A)α). This is because, depending on the specific pH within the range from 3.0 to 11, the aminobenzoic acid is present in the fermentation broth as an anion (H₂NC₆H₃(R)COO⁻) of good water solubility or in the relatively sparingly water-soluble electrically neutral form (H₂NC₆H₃(R)COOH or H₃N⁺C₆H₃(R)COO⁻).

At pH values in the region of 4.7 or less in particular, preferably of 3.7 or less, more preferably of 3.6 or less, the aminobenzoic acid is predominantly to completely in electrically neutral form and therefore crystallizes spontaneously during the fermentation, and so a separate crystallization step is dispensable and the crystallized aminobenzoic acid can be isolated directly from the fermentation broth (case (I)). Case (I) is particularly preferred, for example, when the procedure described gives rise to benefits in the fermentation, such as, in particular, reduced toxicity for the microorganisms in the fermentation broth as a result of the precipitation of the aminobenzoic acid.

In case (I), the fermentation is preferably conducted at a pH in the range from 3.0 to 4.7, preferably 3.2 to 3.7, more preferably 3.4 to 3.6. Step A)α) is then part of the fermentation. In this case, preference is given to using eukaryotes, especially yeasts, as microorganisms. In this regard, reference is made in particular to international patent application WO 2017/102853 A1. Preference is given to using yeast cells capable of converting a fermentable carbon-containing compound to aminobenzoic acid in the presence of a suitable nitrogen source without the aminobenzoic acid thus formed being consumed straight away in intracellular biochemical processes, with the result that aminobenzoic acid accumulates in the cell and is ultimately transferred into the fermentation broth. Eukaryotes such as Ashbya gossypii, Pichia pastoris, Hansenula polymorpha, Kluyveromyces marxianus, Yarrowia lipolytica, Zygosaccharomyces bailii and Saccharomyces cerevisiae are preferably cultivated in an acidic medium and are therefore suitable for case (I) described here. Step A)α) in case (I) is part of the fermentation. After the fermentation has ended, the precipitated aminobenzoic acid is separated off by a (first) solid-liquid phase separation, and is obtained in a mixture with the microorganisms. The procedure for separation of the two may, for example, be such that the aminobenzoic acid is selectively dissolved in maximum concentration (for example with aqueous base solution, aqueous acid solution or an organic solvent) and then separated out of this solution again if required (by another crystallization or concentration). The procedure may also be such that a first portion of the mixture of solids, especially 50%, is taken up in a minimum amount of aqueous base solution (for example to obtain a pH of the supernatant solution of 7.0) and a second portion of the mixture solids, especially 50%, is taken up in a minimum amount of aqueous acid solution (for example to obtain a pH of the supernatant solution of 1.0) and, after the undissolved components have been separated off, the remaining solutions of comparatively high concentration are combined, such that a pH at or close to the isoelectric point is established in the resulting mixture and the aminobenzoic acid precipitates out again. The mother liquor remaining in a (second) solid-liquid phase separation after the precipitated aminobenzoic acid has been separated off has a significantly smaller volume than the mother liquor obtained after the first solid-liquid phase separation and, since it contains only a comparatively small amount of aminobenzoic acid, can be disposed of or advantageously combined with the mother liquor from the first solid-liquid phase separation.

The liquid phase obtained in the first solid-liquid phase separation or the combined liquid phases from the first and second solid-liquid phase separations in this embodiment are the “aqueous solution of the organic acid”. Solid-liquid phase separations in the context of the present invention are always (no matter in what process step) preferably conducted in the form of a filtration, centrifugation or sedimentation, optionally assisted by a preceding flocculation.

At pH values of >4.7 in particular, preferably of 6.0 or more, more preferably of 8.0 or more, the aminobenzoic acid is predominantly to entirely in anionic form, and so a separate crystallization step is conducted (case (II), which is preferred). In case (II), the fermentation is preferably conducted at a pH in the range from >4.7 to 11 (especially 6.0 to 11), preferably 4.8 to 9.0 (especially 6.0 to 9.0), more preferably 5.0 to 8.0 (especially 6.0 to 8.0). In this case, preference is given to using prokaryotes, especially bacteria, as microorganisms. In this regard, particular reference is made here to patent applications WO 2015/124686 A1 and WO 2015/124687 A1, which describe fermentation processes with use of bacteria (see, for example, WO 2015/124687 A1, (i) page 15 line 8 to page 16 line 30, (ii) example 1 (page 29 lines 4 to 26), (iii) example 3 (in particular page 34 lines 10 to 18), (iv) example 4 (in particular page 55 lines 9 to 31) and which afford the anion of aminobenzoic acid as the direct fermentation process product, i.e. are suitable for case (II). In particular, bacteria used are those that are capable of converting a fermentable carbon-containing compound to aminobenzoic acid in the presence of a suitable nitrogen source without the aminobenzoic acid thus formed being consumed straight away in intracellular biochemical processes, with the result that aminobenzoic acid is enriched in the cell and is ultimately transferred into the fermentation broth. Microorganisms such as Corynebacterium glutamicum, Pseudomonas putida or Escherichia coli are preferably cultured at “neutral to basic pH values” and are therefore suitable for case (II) described here. Since the aminobenzoic acid in this case is obtained in the form of the anion, it is crystallized in a step downstream of the fermentation by addition of an inorganic acid, especially hydrochloric acid, sulfuric acid, nitric acid or phosphoric acid, with establishment of a pH in the range from 3.0 to 4.7, preferably 3.2 to 3.7, more preferably 3.4 to 3.6. In this case, step A)α) is thus a step following the fermentation.

### Chemical Methods

In another embodiment, step A) comprises a chemical conversion (i.e. a non-biological method). Particular mention should be made here of the reaction of optionally methyl-substituted phthalimide or phthalamide with an alkali metal hypohalite (especially sodium hypochlorite) in a basic medium. Such methods are known from the literature and are described, for example, in WO 2007/088346 A1. In this case, the desired aminobenzoic acid of the formula (I) is obtained as the anion and is crystallized by addition of an inorganic acid, especially hydrochloric acid, sulfuric acid, nitric acid or phosphoric acid (=step A)α)).

If the crystallization step A)α) is effected separately from the actual preparation, as, for example, in case (II) of fermentative preparation or in the case of a crystallization used for purification of a raw product, as well as the reactor for preparation of the organic acid, an additional industrial apparatus suitable for crystallizations is used, known as a crystallizer in the specialist field. Suitable crystallizers are, for example, stirred tanks or forced circulation crystallizers, such as those of the “Oslo type”. In the crystallizer, the pH is adjusted if required to a suitable value, in the case of the aminobenzoic acid of the formula (I) to a value in the range from 3.0 to 4.7, preferably 3.2 to 3.7, most preferably 3.4 to 3.6. This is preferably accomplished by adding an acid selected from hydrochloric acid, sulfuric acid, nitric acid or phosphoric acid. This adjustment of pH results in conversion of the aminobenzoic acid anions (H₂NC₆H₃(R)COO⁻) predominantly to completely to the electrically neutral form ((H₂NC₆H₃(R) COOH or H₃N+C₆H₃(R)COO⁻) and crystallization thereof. This type of crystallization is also referred to as reactive crystallization. This crystallized aminobenzoic acid is isolated by solid-liquid phase separation, leaving the “aqueous solution of the organic acid”.

### Aliphatic Saturated Dicarboxylic Acids of the Formula (II)

If the aim is to obtain aliphatic saturated dicarboxylic acid of the formula (II), of which hexane-1,6-dicarboxylic acid (=adipic acid) is preferred, it has been found to be useful to establish a pH in the range from 0 to 4.0, preferably 1.8 to 3.0, more preferably 2.0 to 2.5, in step A)α) by addition of an inorganic acid, especially hydrochloric acid, sulfuric acid, nitric acid or phosphoric acid. The exact manner of performance of the crystallization in step A)α) is in turn dependent on the manner of performance of step A) overall. All the possible configurations of the invention described above in association with the preparation of aminobenzoic acids of the formula (I) that are not specific to these aminobenzoic acids are correspondingly also applicable to the case of preparation of aliphatic saturated dicarboxylic acid of the formula (II) which is described hereinafter. This relates, for example, to the configuration of a crystallizer.

### Biological Methods

In one embodiment, step A) comprises a biological method. In a first variant, this is especially conducted by converting a fermentable carbon-containing compound and a nitrogen-containing compound to the desired aliphatic saturated dicarboxylic acid or anion thereof in the presence of microorganisms. Such fermentations have already been described; see in particular the already cited publications Peter C. K. Lau (Editor), Quality Living Through Chemurgy and Green Chemistry, Springer-Verlag GmbH Germany 2016, chapter 3.3, pages 47 to 54, and K. Raj, S. Partow et al., Biocatalytic production of adipic acid from glucose using engineered Saccharomyces cerevisiae in Metabolic Engineering Communications 2018, 6, 28-32.

Suitable microorganisms for a fermentative preparation of an aliphatic saturated dicarboxylic acid of the formula (II) include in particular Escherichia coli, Pseudomonas putida, Corynebacterium glutamicum, Bacillus coagulans, Ashbya gossypii, Pichia pastoris, Hansenula polymorpha, Kluyveromyces marxianus, Yarrowia lipolytica, Zygosaccharomyces bailii or Saccharomyces cerevisiae.

Entirely in analogy with the above-described preparation of the aminobenzoic acid of the formula (I), there is pH dependence here too: Depending on the pH at which the fermentation is conducted, the aliphatic saturated dicarboxylic acid already crystallizes during the fermentation (case (I)), or the fermentation broth contains the aliphatic saturated dicarboxylic acid in the form of its anion, in which case the fermentation is followed by a reactive crystallization (case (II)). In case (I), the fermentation is preferably conducted at a pH in the range from 0 to 4.0, preferably 1.8 to 3.0, more preferably 2.0 to 2.5. The procedure for separation of microorganisms and organic acid may, for example, be such that the saturated aliphatic dicarboxylic acid is selectively dissolved in maximum concentration (for example with aqueous base solution or an organic solvent) and then separated out of this solution again if required (by another crystallization or concentration). Step A)α) is then part of the fermentation. In case (II), the fermentation is preferably conducted at a pH in the range from >4.0 to 11 (especially 6.0 to 11), preferably 5.0 to 9.0 (especially 6.0 to 9.0), more preferably 5.0 to 8.0 (especially 6.0 to 8.0). In this case, the aliphatic saturated dicarboxylic acid is obtained as the anion and is crystallized in a step downstream of the fermentation by addition of an inorganic acid, especially hydrochloric acid, sulfuric acid, nitric acid or phosphoric acid, with establishment of a pH in the range from 0 to 4.0, preferably 1.8 to 3.0, more preferably 2.0 to 2.5. In this case, step A)α) is thus a step following the fermentation.

In a second variant of the preparation of the organic acid using a biological method, enzymes are used for cleavage of polyesters. The enzymatic cleavage of polyesters using cutinases for release of adipic acid has been described, for example, in the following paper: Perz, V., Bleymaier, K., Sinkel, C., Kueper, U., Bonnekessel, M., Ribitsch, D., & Guebitz, G. M. (2016), Substrate specificities of cutinases on aliphatic-aromatic polyesters and on their model substrates, in New biotechnology, 2016, 33 (2), 295-304.

### Chemical Methods

In another embodiment, step A) comprises a chemical conversion (i.e. a non-biological method). Chemical methods for preparation of the dicarboxylic acids of the type specified are known. Reference is made by way of example to the hydrogenation of the corresponding aliphatic unsaturated dicarboxylic acids. In the case of adipic acid, which is most strongly preferred, the preparation can thus be effected by hydrogenation of muconic acid, which is described, for example, in WO2015/086827A1. Step A)α) is conducted here in particular for purification of the product obtained in the hydrogenation. An alternative means of preparation of adipic acid is the oxidation, already mentioned at the outset and described many times in the literature, of cyclohexane with oxygen or oxygenous gas mixtures (especially air) to cyclohexanol and cyclohexanone, and subsequent oxidation thereof with nitric acid (see A. Castellan et al. in Catal. Today 1991, 9, 237-254 for a review).

In the case of a chemical conversion too, step A) may comprise the cleavage of polyesters, for example by acidic or alkaline hydrolysis. In addition, step A) may comprise the acidic or alkaline hydrolysis of a polyamide based on hexamethylenediamine and adipic acid, as described, for example, in U.S. Pat. No. 3,223,731.

### Optional Step of Depleting the Fraction of the Inorganic Acid Dissolved in the First Aqueous Solution of the Organic Acid

The first aqueous solution of the organic acid obtained by one of the methods described above can be fed directly to step B). However, it is likewise possible already to reduce this concentration of organic acid in this first aqueous solution of organic acid prior to performance of step B) in another way. Suitable methods for this purpose are the methods of extraction and adsorption that are known in the specialist field.

In the case of use of an extractive method, the first aqueous solution of the organic acid is extracted with an organic extractant, which is followed by a liquid-liquid phase separation to obtain an aqueous phase (=organic acid-depleted second aqueous solution of the organic acid) and an organic phase (=organic acid-laden extractant). Liquid-liquid phase separations in the context of the present invention are always preferably conducted (no matter in which process step) by means of separators, decanters or centrifuges.

Suitable extractants are, for example, (i) alcohols, especially alkanols, having 4 to 12 carbon atoms (especially 1-decanol and/or 1-dodecanol), preferably 8 to 12 carbon atoms, more preferably 9 to 11 carbon atoms, (ii) ketones (especially methyl iso-butyl ketone and/or cyclohexanone), (iii) ethers (especially diethyl ether), (iv) esters (especially butyl acetate), (v) aromatic, optionally halogen-substituted hydrocarbons (especially benzene, toluene, monochlorobenzene and/or dichlorobenzene) and (vi) mixtures of two or more of the aforementioned extractants,

Suitable apparatuses for performance of an extraction are known in the specialist field. Suitable examples include what are called mixer-settlers or extraction columns having a number of theoretical plates of preferably 3 to 10, more preferably 3 to 7, most preferably 4 to 6. The mass-based phase ratio of the organic phase to the aqueous phase is preferably 0.20 to 1.0, more preferably 0.20 to 0.50, most preferably 0.35 to 0.50. After separation of the organic solvent phase and the aqueous phase, the aqueous solution of the organic acid is fed to step B). The organic acid-laden extractant is preferably sent to an back-extraction in which the organic acid is converted to the anion by extraction with an aqueous base solution and, after phase separation, an aqueous phase containing anions of the organic acid and an alcoholic phase is obtained. Since impurities can occur in the preparation of the organic acid that are comparatively polar, and these comparatively polar impurities in particular can be transferred to the aqueous phase at least in fractions in this step, it is preferable to take measures for discharge of such impurities. The extraction can be conducted at temperatures of 20° C. to 90° C., preferably 25° C. to 70° C., more preferably 30° C. to 50° C., especially also at ambient temperature.

The aqueous base solution used for the re-extraction is preferably an aqueous solution of an alkali metal or alkaline earth metal hydroxide (especially sodium hydroxide solution, potassium hydroxide solution or calcium hydroxide solution), of an alkali metal or alkaline earth metal hydrogencarbonate (especially a sodium or potassium hydrogencarbonate solution), of an alkali metal or alkaline earth metal carbonate (especially a sodium or potassium carbonate solution) or a mixture of two or more of the aforementioned compounds. Sodium hydroxide solution and potassium hydroxide solution are particularly preferred. Irrespective of the base used, it is preferable to observe a molar ratio of hydroxide ions to organic acid group of 1.0 to 5.0, preferably 1.0 to 2.0 and more preferably 1.0 to 1.5.

Suitable apparatuses for this re-extraction are the same as described above for the extraction. The mass-based phase ratio of the organic phase to the aqueous phase is preferably 0.20 to 1.0, more preferably 0.20 to 0.50, most preferably 0.20 to 0.40.

Rather than an extraction, it is also possible to use an adsorption in order to deplete the first aqueous solution of organic acid. For this purpose, the first aqueous solution is passed through a bed of an adsorbent on which the organic acid is adsorbed. Suitable adsorbents are typically nonpolar adsorbents, for example (i) activated carbon, (ii) graphite or (iii) adsorber polymers, but also (typically) (iv) polar adsorbents, for example silica gel, limestone or aluminium silicates, and (v) mixtures of two or more of the aforementioned adsorbents. Adsorption is preferably effected by flow of the aqueous solution through the adsorbent until the adsorption properties of the adsorbent decline and regeneration of the adsorbent by desorption of the inorganic acid becomes necessary. Desorption is preferably effected with aqueous solutions of pH in the range from 5 to 10 (adjustment of pH, for example, with sodium hydroxide solution or hydrochloric acid), or alternatively with organic solvents, especially 1-dodecanol. A variant of such a workup by a sequence of an adsorption step and desorption step is described in international application WO 2018/114841 A1. The process disclosed therein is more particularly characterized in that the desorption is conducted in an acidic environment (pH-0.8 to 3.0). In this embodiment, the liquid phase leaving the bed of the adsorbent in the course of desorption is the second aqueous solution of organic acid and is fed to step B).

Suitable apparatuses for performance of an adsorption are known in the specialist field. Suitable examples are columns, and on the laboratory scale appropriately chromatography columns with which the adsorbent is charged, especially in the form of an adsorber bed.

### Metal Salt Treatment

In step B) of the process of the invention, the first or second aqueous solution of the organic acid obtained in step A) is treated with a metal salt, the metal ions of which comprise Mn²⁺ Fe²⁺ and/or Cu²⁺ and are especially selected from the group consisting of Mn²⁺, Fe²⁺ and Cu²⁺, with precipitation of a metal compound of the organic acid (sparingly soluble complex or sparingly soluble salt), which is followed by removal of the precipitated metal compound of the organic acid by a solid-liquid phase separation. Of the metal ions usable in accordance with the invention, Fe²⁺ and Cu²⁺ are preferred (especially in the case of obtaining aliphatic saturated dicarboxylic acids); particular preference is given to Cu²⁺ (again in particular in the case of obtaining aliphatic saturated dicarboxylic acids).

The optimal pH in step B) is guided by the type of organic acid to be obtained and, in the case of the aminobenzoic acids of the formula (I), is preferably 3.0 to 4.7, more preferably 3.2 to 3.7, most preferably 3.4 to 3.6, and, in the case of the aliphatic saturated dicarboxylic acids of the formula (II), is preferably >4.0 to 7.0, more preferably 4.7 to 5.8, most preferably 5.3 to 5.7. If the pH should threaten to deviate from the target ranges owing to acidic or basic properties of the metal salt added, this can be easily compensated for by addition of acid (preferably hydrochloric acid, sulfuric acid, nitric acid or phosphoric acid) or base (preferably sodium hydroxide solution, potassium hydroxide solution or calcium hydroxide).

The metal salt to be used in step B) is preferably a sulfate, chloride and/or hydroxide. The use of a hydroxide enables a particularly economically viable configuration of the process of the invention in which the precipitated metal hydroxide obtained in step C) described further down is returned to step B), where it is used as a constituent of the metal salt. Any excessive rise in pH in step B) can be countered by addition of acid.

The metal ions of the metal salt are preferably added in not more than a stoichiometric amount, based on the amount of the organic acid.

The metal salt treatment is preferably conducted at 20° C. to 50° C., more preferably 20° C. to 30° C., especially also at ambient temperature. An example of a suitable reaction apparatus is a stirred tank reactor (or two or more stirred tank reactors, especially connected in series). The procedure for performance of the treatment may be such that the metal salt (e.g. Cu(OH)₂) is initially charged in the reactor and admixed with the aqueous solution of the organic acid, if required with active adjustment of pH (see the above details relating to suitable pH values). After precipitation of the sparingly soluble metal compound of the organic acid, it is separated off by solid-liquid phase separation. The remaining aqueous phase can be disposed of as wastewater. Since this aqueous phase still contains metal salts in fractions corresponding to their solubility at the present (acidic; see above) pH, and these proportions may be undesirably high from an environmental point of view, in a preferred embodiment of the invention, the pH of the aqueous liquid phase obtained in the solid-liquid phase separation should be increased to values of 8.0 to 14, preferably 10 to 13, in order to precipitate dissolved metal ions as sparingly soluble hydroxides. These can then be separated off in a second solid-liquid phase separation and recycled into step B). The aqueous liquid phase obtained in the second solid-liquid phase separation can then be disposed of without difficulty, for example in a biological treatment plant. Suitable bases for increasing the pH are standard bases such as alkali metal and alkaline earth metal hydroxides, alkali metal and alkaline earth metal carbonates, or alkali metal and alkaline earth metal hydrogencarbonates, especially sodium hydroxide solution, potassium hydroxide solution or calcium hydroxide solution.

### Base Treatment

In step C) of the process of the invention, base treatment of the metal compound of the organic acid obtained in step B) is effected. The organic acid goes into solution here in anionic form, and the metal ions used precipitate out as hydroxides.

It is preferable to conduct step C) in multiple stages in n steps conducted in succession, where n is a natural number in the range from 2 to 10, preferably 3 to 9, more preferably 4 to 8. For this purpose, in each of the n steps, a portion of the metal compound of the organic acid that has been removed is treated with an aqueous base solution with precipitation of metal hydroxide, wherein each step is followed by removal of precipitated metal hydroxide by solid-liquid phase separation to leave a basic aqueous solution containing anions of the organic acid, wherein the aqueous base solution in the second to nth step comprises the basic aqueous solution containing anions of the organic acid that has been obtained in the respective preceding step (and optionally consists thereof, i.e. does not comprise any further constituents). The crystallization according to step D) which will be described in detail hereinafter is conducted with the basic aqueous solution containing anions of the organic acid that has been obtained in the nth step of C).

In step C), it is preferable to observe a pH in the range from 9.0 to 14, preferably 10 to 14 and more preferably 11 to 13, with further metered addition of base solution if required. Suitable aqueous base solutions are all bases familiar to the person skilled in the art, such as, in particular, aqueous solutions of alkali metal or alkaline earth metal hydroxides (especially sodium hydroxide solution, potassium hydroxide solution or calcium hydroxide solution (aqueous solution of calcium hydroxide)), alkali metal or alkaline earth metal hydrogencarbonates, alkali metal or alkaline earth metal carbonates (especially a sodium or potassium hydrogencarbonate solution or a sodium or potassium carbonate solution) or a mixture of two or more of the aforementioned base solutions. Sodium hydroxide solution and potassium hydroxide solution are particularly preferred. In the case of multistage performance of step C), the pH values mentioned are observed in each of the n steps.

Step C), like step B), is preferably conducted at ambient temperature. Here too, a stirred tank reactor is suitable (or two or more stirred tank reactors, especially connected in series).

### Crystallization

In step D), the anions of the organic acid present in the basic aqueous solution obtained in step C) are crystallized. This is accomplished by lowering the pH by adding an inorganic acid, especially hydrochloric acid, sulfuric acid, nitric acid or phosphoric acid. The simplest means of performing step D) is to conduct the basic aqueous solution obtained in step C) into the crystallization according to A)α), where corresponding acidic conditions already exist. However, it is also of course possible to conduct step D) separately from A)α), for instance in a separate crystallizer. Suitable types for this purpose are those as described above, optionally (because of the smaller amounts) in a smaller design. The pH values should then be adjusted as described further above for A)α).

The appended figures are intended to deepen understanding of the invention.

FIG. 1 shows a preferred embodiment of the process of the invention in which step Aa) is a step downstream of the actual preparation of the organic acid and in which step D) is conducted, in which the aqueous solution containing anions of the organic acid from step C) is returned to the crystallization according to Aa).

The reference numerals have the following meanings:

| | |
|---|---|
| 1000 | crystallizer for performance of the crystallization in step Aα); |
| 2000 | reactor, e.g. stirred tank reactor, for performance of the metal salt treatment in step B); |
| 3000 | apparatus, e.g. filtration unit or centrifuge, for solid-liquid separation in step B); |
| 4000 | reactor, e.g. stirred tank reactor, for performance of the base treatment in step C); |
| 5000 | apparatus, e.g. filtration unit or centrifuge, for solid-liquid separation in step C); |
| (1) | aqueous medium from the preparation of the organic acid in step A) (for example a |
| | neutral to basic fermentation broth that has been freed of microorganisms); |
| (2) | optionally, in the case of performance of step Aα) as a reactive crystallization: |
| | compound that triggers crystallization (for example an acid such as hydrochloric acid in |
| | order to protonate and hence precipitate the organic acid dissolved in anionic form in |
| | the neutral to basic fermentation broth that has been freed of microorganisms); |
| (3) | organic acid crystallized in step Aα); |
| (4) | aqueous solution of the organic acid (mother liquor from the crystallization in step Aα)); |
| (5) | aqueous acid or base for adjustment of the pH in the metal salt solution in step B) |
| | (when an acidic metal salt is fed in via (6), an aqueous base is first fed in via (5); after |
| | establishment of the recycling of precipitated metal hydroxide via (12), the feed of base |
| | is replaced by feeding of an aqueous acid); |
| (6) | (fresh, nonrecycled) metal salt; |
| (7) | suspension of precipitated metal compound of the organic acid; |
| (8) | aqueous liquid phase obtained in the solid-liquid separation; |
| (9) | metal compound of the organic acid; |
| (10) | aqueous base solution, e.g. sodium hydroxide solution; |
| (11) | suspension of precipitated metal hydroxide in aqueous solution containing anions of |
| | the organic acid; |
| (12) | metal hydroxide; is returned in this embodiment to the metal salt treatment in step B); |
| (13) | basic aqueous solution containing anions of the organic acids; is returned in this |
| | embodiment, for performance of step D), into the crystallization in step Aα). |

FIG. 2 shows the preferred configuration of step C) in n stages (here by way of example with n=7). Here, the metal compound of the organic acid (9) obtained in step B) is divided into seven portions (9.1, 9.2, . . . 9.7), the first of which (9.1) is treated with aqueous base solution (10), and, after solid-liquid phase separation, a first portion (12.1) of precipitated metal hydroxide (12) is obtained and removed (for the sake of simplicity, the steps of base treatment and solid-liquid phase separation are not described separately here). The remaining basic aqueous liquid phase is added to the second portion (9.2) of the metal compound, and another solid-liquid phase separation is conducted, in which a second portion (12.2) of precipitated metal hydroxide (12) is removed. After these steps have been conducted with five further portions of the metal compound of the organic acid, the basic aqueous solution containing anions of the organic acid (13) is finally obtained. The resultant portions (12.1, 12.2, . . . 12.7) of precipitated metal hydroxide are preferably combined and then fed as shown in FIG. 1 to the metal salt treatment in step B).

### EXAMPLES

The examples that follow proceeded from organic acids that were not produced specially for the examples but sourced from commercial sources. The aqueous solution of the organic acid obtained in step A) of the method of the invention was therefore made up artificially, but this is not detrimental to the meaningfulness of the experiments.

### Example 1: Precipitation of 2-Aminobenzoic Acid in Aqueous Solution by Addition of CuSO₄, FeSO₄ or MnSO₄

### Chemicals:

2-Aminobenzoic acid (anthranilic acid, ortho-aminobenzoic acid; CAS No: 118-92-3) was sourced from Sigma-Aldrich with a purity of >98%, 2-Aminobenzoic acid is abbreviated hereinafter to “oAB”, regardless of the form in which it exists (electrically neutral or as the anion). CuSO₄, FeSO₄·7 H₂O and MnSO₄·H₂O were sourced from Sigma-Aldrich with a purity of >99%. Ultrapure water (prepared with a Millipore laboratory water treatment system) was used. NaOH (pellets) and HCl_{(aq)} (37% strength) were used from Acros.

### Experiment:

### a) Providing the Aqueous Solution of the Organic Acid

By dissolving oAB in water, an aqueous oAB solution (oAB concentration 5 g/L) was prepared and, in this way, an aqueous solution of the organic acid obtained in a crystallization in step A)α) (=mother liquor of the crystallization) was made up artificially.

b) Treating the Aqueous Solution of the Organic Acid with a Metal Salt (Step B))

The aqueous oAB solution and a metal salt (CuSO₄, FeSO₄ or MnSO₄) were mixed at room temperature for at least 30 minutes (molar ratio of metal salt:oAB=1:2). The pH was adjusted to about 3.5 with a 1M NaOH solution (corresponding to the isoelectric point of oAB). This was followed by a wait period of 30 minutes to enable complete settling of the solid formed (metal compound of the organic acid). A sample of the aqueous supernatant was analyzed by HPLC (high-performance liquid chromatography) with regard to the oAB concentration (sample A). The solid (Cu(oAB)₂, Fe(oAB)₂ and/or Mn(oAB)₂) was separated from the liquid phase by centrifugation.

### c) Base Treatment of the Metal Compound of the Organic Acid Removed (Step C))

In the case of the experiments with Fe and Cu, the solid removed was resuspended in water, and the pH was adjusted to pH=12.0 to 12.5 with a 1M NaOH solution. The oAB-metal compound went into solution, and metal hydroxide (Cu(OH)₂ and/or Fe(OH)₂) precipitated out in solid form. After mixing for 30 minutes and a settling time of 30 minutes, a sample of the aqueous supernatant was analyzed for oAB by HPLC (sample B). The solid (precipitated metal hydroxide) was subsequently separated from the liquid phase (basic aqueous solution containing anions of the organic acid). In the case of iron(II) hydroxide, the base treatment was conducted repeatedly (see FIG. 2):

By repeated addition of Fe (oAB) 2 (seven additions in total), the intention was to further increase the oAB concentration in the liquid phase. In each of the seven steps, Fe(OH)₂ precipitated out in solid form, and was removed by centrifugation after each addition.

d) Recycling of Precipitated Metal Hydroxide into the Step of Treating the Aqueous Solution of the Organic Acid with a Metal Salt

Aqueous oAB solution (5 g/L) was mixed with the solid metal hydroxide at pH=3.5 for 30 minutes. The metal hydroxide dissolved, and Cu(oAB)₂ and/or Fe(oAB)₂ precipitated out. After the solids had settled out, a sample of the liquid phase was analyzed with regard to the oAB content with the aid of HPLC analysis (sample C).

### High-Performance Liquid Chromatography:

An Agilent 1260 Infinity II LC with diode array detector was used for the quantification of oAB. The stationary phase used was a C18 Zorbax Eclipse (4.6×150 mm) with pre-column. The mobile phase used was water with 0.1% H₃PO₄ (A) and methanol (B) with the following linear gradient:
Min 0: 95% A/5% B
Min 4: 95% A/5% B
Min 15: 2% A/98% B
Min 16: 2% A/98% B
Min 16.5: 95% A/5% B
Min 21: 95% A/5% B

The volume flow rate was set at 0.5 ml/min. 5 μL of sample was injected each time. The column temperature was 35° C. oAB was detected at a wavelength of 254 nm and 330 nm and compared with a calibration curve.

### Results:

The aqueous supernatant after precipitation of the metal compound of the organic acid (sample A from b)) had an oAB concentration below the detection limit of 0.05 g_{oAB}/L. This shows that it was possible to precipitate oAB in solid form virtually quantitatively (>99%) in the form of an oAB-metal compound.

The aqueous supernatant after the precipitation of metal hydroxide triggered by base treatment (sample B from c)) showed an oAB concentration of 14 g/L. In the case of iron, by virtue of the performance of the base treatment in seven steps, it was possible to increase the oAB concentration in the liquid phase to 301 g/L.

It was shown that the solid (precipitated metal hydroxide, Cu(OH)₂ and/or Fe(OH)₂) can be used again for complexation of oAB from aqueous solution (see d)). Analogously to sample A from b), in sample C after addition both of Fe(OH)₂ and Cu(OH)₂, an oAB concentration below the detection limit of 0.05 g_{oAB}/L was measured.

Overall, it can be stated that it was possible to distinctly concentrate the oAB present in the original “aqueous solution of the organic acid” (5 g/L) by the inventive sequence of treatment with a metal salt and base treatment (to at least 14 g/L, in the case of base treatment in seven steps to 301 g/L). The distinctly more concentrated solutions (of the anion of oAB) thus obtained can be subjected without any problems to a crystallization step for the purpose of isolation of oAB (step D)).

### Example 2: Precipitation of Adipic Acid in Aqueous Solution by Addition of CuSO₄

### Chemicals:

Adipic acid (CAS No: 124-04-9) was sourced with a purity of 98%. CuSO₄ was sourced from Sigma-Aldrich with a purity of >99%. Ultrapure water (prepared with a Millipore laboratory water treatment system) was used. NaOH (pellets) and HCl_{(aq)} (37% strength) were used from Acros.

### Experiment:

### a) Providing the Aqueous Solution of the Organic Acid

By dissolving adipic acid in water, an aqueous adipic acid solution (12 g/L) was prepared and, in this way, an aqueous solution of the organic acid obtained in a crystallization in step A)α) (=mother liquor of the crystallization) was made up artificially.

b) Treating the Aqueous Solution of the Organic Acid with a Metal Salt (Step B))

The aqueous adipic acid solution and a metal salt (CuSO₄) were mixed at room temperature for at least 30 minutes (molar ratio of metal salt:adipic acid=1:1). The pH was adjusted to about 5.5 with a 1M NaOH solution. This was followed by a wait period of 30 minutes to enable complete settling of the solid formed (metal compound of the organic acid). A sample of the aqueous supernatant was analyzed by HPLC (high-performance liquid chromatography) with regard to the adipic acid concentration (sample A).

The solid (copper adipate) was separated from the liquid phase by centrifugation.

### c) Base Treatment of the Metal Compound of the Organic Acid Removed (Step C))

The solids removed were resuspended in water, and the pH was adjusted to 12 to 12.5 with a 1M NaOH solution. The adipic acid-metal compound goes into solution, and metal hydroxide (Cu(OH)₂) precipitates out. After mixing for 30 minutes and a settling time of 30 minutes, a sample of the aqueous supernatant was analyzed for the adipic acid content by HPLC (sample B).

The solid (precipitated metal hydroxide) was subsequently separated from the liquid phase (basic aqueous solution containing anions of the organic acid).

d) Use of Precipitated Metal Hydroxide in the Step of Treating the Aqueous Solution of the Organic Acid with a Metal Salt

Aqueous adipic acid solution (12 g/L) was mixed with the metal hydroxide (roughly stoichiometrically) at pH=4.5 for 30 minutes. The metal hydroxide dissolved, and metal adipate precipitated out. After the solids had settled out, a sample of the liquid phase was analyzed with regard to the adipic acid content with the aid of HPLC analysis (sample C).

### High-Performance Liquid Chromatography:

The samples were prepared in a 0.1M aqueous phosphate buffer with 0.1% by weight of pimelic acid.

An Agilent 1260 Infinity II LC with diode array detector was used for the quantification of adipic acid. The stationary phase used was a C18 Zorbax Eclipse (4.6×150 mm) with pre-column (Poroshell 120 EC-C18 (4.6×5 mm)). The mobile phase used was water with 0.1% H₃PO₄ (A) and acetonitrile (B) with the following linear gradient:
Min 0: 95% A/5% B
Min 0.5: 95% A/5% B
Min 4.5: 75% A/25% B
Min 6.5: 75% A/25% B
Min 6.6.5: 95% A/5% B
Min 9.5: 95% A/5% B

The volume flow rate was set at 1.5 mL/min. 1 μL of sample was injected each time. The column temperature was 35° C. Adipic acid was detected at a wavelength of 208.8 nm and compared with a calibration curve.

### Results:

The aqueous supernatant after precipitation of the metal compound of the organic acid (sample A from b)) had an adipic acid concentration of 0.99 g/L. This shows the low solubility of the salt.

The aqueous supernatant after the precipitation of metal hydroxide triggered by base treatment (sample B from c)) showed an adipic acid concentration of 24 g/L.

It was shown that the solid (precipitated Cu(OH)₂) can be used again for precipitation of adipic acid from aqueous solution (see d)). The adipic acid concentration in sample C from d) was 3 g/L. It is to be expected on the basis of the present data that this value can still be reduced in the case of performance of d) at pH=5.5.

Overall, it can be stated that it was possible to distinctly concentrate the adipic acid present in the original “aqueous solution of the organic acid” (12 g/L) by the inventive sequence of treatment with a metal salt and base treatment (to at least 24 g/L). The distinctly more concentrated (adipate) solutions thus obtained can be subjected without any problems to a crystallization step for the purpose of isolation of adipic acid (step D)).

### Example 3: Precipitation of 2-Amino-5-Methylbenzoic Acid in Aqueous Solution by Addition of CuSO₄

### Chemicals:

2-Amino-5-methylbenzoic acid (CAS No: 2941-78-8) was sourced from Sigma-Aldrich with a purity of 97%. CuSO₄ was sourced from Sigma-Aldrich with a purity of >99%. Ultrapure water (prepared with a Millipore laboratory water treatment system) was used. NaOH (pellets) and HCl_{(aq)} (37% strength) were used from Acros.

### Experiment:

### a) Providing the Aqueous Solution of the Organic Acid

By dissolving 2-amino-5-methylbenzoic acid in water, an aqueous 2-amino-5-methylbenzoic acid solution (1 g/L) was prepared and, in this way, an aqueous solution of the organic acid obtained in a crystallization in step A)α) (=mother liquor of the crystallization) was made up artificially.

b) Treating the Aqueous Solution of the Organic Acid with a Metal Salt (Step B))

The aqueous 2-amino-5-methylbenzoic acid solution and a metal salt (CuSO₄) were mixed at room temperature for at least 30 minutes (molar ratio of metal salt:2-amino-5-methylbenzoic acid=1:1). The pH was set to 3.5 with NaOH. This was followed by a wait period of 30 minutes to enable complete settling of the solid formed (metal compound of the organic acid). A sample of the aqueous supernatant was analyzed by HPLC (high-performance liquid chromatography) with regard to the 2-amino-5-methylbenzoic acid concentration (sample A).

The solid (copper 2-amino-5-methylbenzoate) was separated from the liquid phase. A centrifuge was utilized for better solid-liquid separation.

### c) Base Treatment of the Metal Compound of the Organic Acid Removed (Step C))

The solids removed were resuspended in water, and the pH was adjusted to 12.5 with a 1M NaOH solution. The 2-amino-5-methylbenzoate-metal compound goes into solution, and metal hydroxide (Cu(OH)₂) precipitates out. After mixing for 30 minutes and a settling time of 30 minutes, a sample of the aqueous supernatant was analyzed for the 2-amino-5-methylbenzoic acid content by HPLC (sample B).

### High-Performance Liquid Chromatography:

An Agilent 1260 Infinity II LC with diode array detector was used for the quantification of 2-amino-5-methylbenzoic acid. The stationary phase used was a C18 Zorbax Eclipse (4.6×150 mm) with pre-column. The mobile phase used was water with 0.1% H₃PO₄ (A) and methanol (B) with the following linear gradient:
Min 0: 95% A/5% B
Min 4: 95% A/5% B
Min 15: 2% A/98% B
Min 16: 2% A/98% B
Min 16.5: 95% A/5% B
Min 21: 95% A/5% B

The volume flow rate was set at 0.5 ml/min. 5 μL of sample was injected each time. The column temperature was 35° C. 2-Amino-5-methylbenzoic acid was detected at a wavelength of 254 nm and 330 nm and compared with a calibration curve.

### Results:

The aqueous supernatant after precipitation of the metal compound of the organic acid (sample A from b)) had a 2-amino-5-methylbenzoic acid concentration of less than 0.01 g/L. This shows the low solubility of the metal compound.

The aqueous supernatant after the precipitation of metal hydroxide triggered by base treatment (sample B from c)) showed a 2-amino-5-methylbenzoic acid concentration of 0.72 g/L.

Overall, it can be stated that it was possible to separate the 2-amino-5-methylbenzoic acid present in the original “aqueous solution of the organic acid” (1 g/L) from the aqueous solution and then redissolve it in water by the inventive sequence of treatment with a metal salt and base treatment. It is to be expected that a distinct concentration of the 2-amino-5-methylbenzoic acid concentration (analogously to example 1) is achievable. No concentration was conducted in this example. The (2-amino-5-methylbenzoic acid) solutions thus obtained can be subjected without any problems to a crystallization step for the purpose of isolation of 2-amino-5-methylbenzoic acid (step D)).

1. A process for obtaining an organic acid from an aqueous solution of the organic acid, wherein the organic acid comprises:
(i) an aminobenzoic acid of the formula 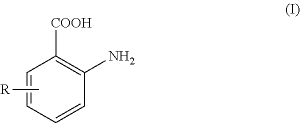
in which R is CH₃ or H,
or
(ii) an aliphatic saturated dicarboxylic acid of the formula 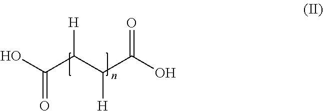
in which n=1 or 2,
said process comprising:
A) preparing the organic acid by a biological process or a chemical reaction, wherein
α) the organic acid is crystallized from an aqueous medium during or after the preparation, followed by
1) removing the fractions of the organic acid that have precipitated in the crystallization by a solid-liquid phase separation to leave a first aqueous solution of the organic acid, and
2) optionally depleting the fraction of the inorganic acid dissolved in the first aqueous solution of the organic acid by extracting organic acid with an organic extractant or by adsorbing organic acid on an adsorbent, followed by removing the organic extractant or adsorbent to obtain a second aqueous solution of the organic acid;
B) treating the first aqueous solution of the organic acid or the second aqueous solution of the organic acid with a metal salt, the metal ions of which include Mn²⁺, Fe²⁺ and/or Cu²⁺, to precipitate a metal compound of the organic acid, followed by removing of the precipitated metal compound of the organic acid by a solid-liquid phase separation;
C) base treatment of the metal compound of the organic acid that has been removed with an aqueous base solution with precipitation of metal hydroxide and removal of precipitated metal hydroxide by a solid-liquid phase separation to leave a basic aqueous solution containing anions of the organic acid; and
D) crystallizing organic acid out of the basic aqueous solution containing anions of the organic acid that has been obtained in C) by addition of an inorganic acid.

2. The process as claimed in claim 1, in which the base treatment in C) comprises n steps conducted in succession, where n is a natural number in the range from 2 to 10, wherein
in each of the n steps, a portion of the metal compound of the organic acid that has been removed is treated with an aqueous base solution with precipitation of metal hydroxide, wherein each step is followed by removal of precipitated metal hydroxide by solid-liquid phase separation to leave a basic aqueous solution containing anions of the organic acid, wherein the aqueous base solution in the second to nth step comprises the basic aqueous solution containing anions of the organic acid that has been obtained in the preceding step;
wherein the crystallizing of the organic acid in D) is conducted with the basic aqueous solution containing anions of the organic acid that has been obtained in the nth step of C).

3. The process as claimed in claim 1,
in which the crystallizing of organic acid in D) includes the recycling of the basic aqueous solution comprising anions of the organic acid that has been obtained in C) to A)α);
or
in which the crystallizing of organic acid in D) is conducted separately from A)α).

4. The process as claimed in claim 1, in which the metal hydroxide precipitated in C) is recycled into step B), where it is used as a constituent of the metal salt.

5. The process as claimed in claim 1, in which the organic acid is the aminobenzoic acid of the formula (I).

6. (canceled)

7. The process as claimed in claim 5, in which in A)α) a pH in the range from 3.0 to 4.7 is established by addition of an inorganic acid, and a pH within the same range is maintained in B).

8. The process as claimed in claim 5, in which A) comprises a biological process comprising the fermentation of a raw material containing a fermentable carbon-containing compound and a nitrogen-containing compound in the presence of microorganisms or in which A) comprises a chemical conversion.

9-10. (canceled)

11. The process as claimed in claim 1, in which the organic acid is the aliphatic saturated dicarboxylic acid of the formula (II).

12. (canceled)

13. The process as claimed in claim 11, in which in A)α) a pH in the range from 0 to 4.0 is established by addition of an inorganic acid, and in B) a pH in the range from >4.0 to 7.0 is established by addition of a base.

14. The process as claimed in claim 11, in which A) comprises a biological process comprising the fermentation of a raw material containing a fermentable carbon-containing compound in the presence of microorganisms, or in which A) comprises a chemical conversion.

15-18. (canceled)

19. The process as claimed in claim 1, in which the metal ions of the metal salt used in B) comprise Fe²⁺ and/or Cu²⁺.

20. The process as claimed in claim 1, in which a pH in the range from 9.0 to 14 is established in C).

21. The process as claimed in claim 1, in which the metal ions are added in not more than a stoichiometric amount in B), based on the amount of the inorganic acid.

22. The process as claimed in claim 1, in which the metal salt comprises a sulfate, chloride and/or hydroxide.

23. The process as claimed in claim 1, in which the removal of the precipitated metal compound of the organic acid by solid-liquid phase separation in B) leaves an aqueous phase which is adjusted to a pH in the range from 8.0 to 14 by addition of a base, with precipitation of metal hydroxides that are removed by a further solid-liquid phase separation, in which the metal hydroxides are used as a constituent of the metal salt in B).
